(19) United States
(12) Patent Application Publication
(10) Pub. No.: US 2026/0192113 A1
(43) Pub. Date: Jul. 9, 2026

(54) METHOD OF TREATING CARDIAC ARRHYTHMIA USING ELECTRODE APPARATUS

(71) Applicant: DEEPQURE INC., Seoul (KR)

(72) Inventors: Chanho PARK, Seoul (KR); Du-Jin BACH, Seoul (KR); Youngil YOON, Seoul (KR); Keewan KIM, Seoul (KR); Jongsun PARK, Seoul (KR); Eun-Keun CHOI, Seoul (KR); Soonil KWON, Seoul (KR); Mookang KIM, Seoul (KR)

(21) Appl. No.: 19/554,707

(22) Filed: Mar. 3, 2026

Related U.S. Application Data
(63) Continuation of application No. PCT/KR2023/013193, filed on Sep. 4, 2023.

(51) Int. Cl. A61N 1/36

(52) U.S. Cl. CPC A61N 1/36114

(57) ABSTRACT

A method of treating cardiac arrhythmia using an electrode apparatus provided herein includes: a process of inserting the electrode apparatus into a body via laparoscopy; a process of enclosing a tube in the body with an electrode unit of the electrode apparatus by controlling a manipulation unit of the electrode apparatus; a process of blocking a sympathetic nerve tissue by transferring energy to the tube in the body for a predetermined period of time through the electrode unit; and a process of restoring to a state before the electrode unit encloses the tube in the body by controlling the manipulation unit.

### TECHNICAL FIELD

The present disclosure relates to a method of treating cardiac arrhythmia using an electrode apparatus.

### BACKGROUND

In general, atrial fibrillation (AF) is a type of arrhythmia that causes irregular heartbeats due to rapid heartbeats originating in the atria. It can be treated by methods, such as medication, pulmonary vein isolation through radiofrequency catheter ablation or cryoballoon ablation, and surgery with the Maze procedure.

When attempting to restore normal heart rhythm through medication, it is difficult to expect a complete cure for AF with the goal to alleviate symptoms. When the Maze procedure is used for surgery, the function of the left atrium may not be fully preserved after the surgery. Therefore, recently, minimally invasive arrhythmia surgery, which can be performed via thoracoscopy, has been developed.

Meanwhile, denervation refers to a procedure of damaging a specific nerve to control an autonomic nervous system that is abnormally and excessively activated. For example, renal denervation may treat hypertension and cardiac diseases by damaging renal sympathetic nerves directed to the kidneys, and pulmonary denervation may treat pulmonary diseases by damaging parasympathetic nerves directed to the lungs.

Nerves generally surround an outer wall of a tube, such as a blood vessel or a bronchus. In such a case, it may be necessary to measure nerve signals by surrounding the outer wall of the tube, to deliver electrical stimulation to the corresponding nerve, or to deliver various types of energy to damage or destroy the nerve.

For example, when a procedure is performed on a renal artery, a diameter of a main renal artery, which becomes a procedure target, is about 5 to 7 mm, and an accessory renal artery having a diameter of about 1 to 2 mm may also be a procedure target. Also, a tube on which nerves are distributed may have various sizes depending on individuals, and the size may vary depending on a location.

In performing such a procedure, it is important to accurately position a component including an electrode formed at a distal end of a catheter so as to surround an outer wall of the tube. Specifically, in order to effectively block or regulate a nerve, the outer wall of the tube on which the nerve is distributed should be surrounded in a circumferential direction, and an operation of disposing the component having the electrode formed thereon in a state of surrounding the tube is required to be reliably and rapidly performed. In particular, it is important to safely bring the component having the electrode formed thereon into close contact with the outer wall of the tube in the body so as not to damage the tube in the body that may be easily damaged by external stimulation.

Korean Patent Publication No. 10-2013-0108401 (published on Oct. 2, 2013) is disclosed.

### DISCLOSURE OF THE INVENTION

### Problems to be Solved by the Invention

The present disclosure is conceived to provide a method of treating cardiac arrhythmia by inserting an electrode apparatus into a body via laparoscopy and enclosing a tube in the body with an electrode unit of the electrode apparatus to block a sympathetic nerve tissue.

However, the problems to be solved by the present disclosure are not limited to the above-described problems. Although not described herein, other problems to be solved by the present disclosure can be clearly understood by a person with ordinary skill in the art from the following descriptions.

### Means for Solving the Problems

According to an aspect of the present disclosure, a method of treating cardiac arrhythmia using an electrode apparatus includes: a process of inserting the electrode apparatus into a body via laparoscopy; a process of enclosing a tube in the body with an electrode unit of the electrode apparatus by controlling a manipulation unit of the electrode apparatus; a process of blocking a sympathetic nerve tissue by transferring energy to the tube in the body for a predetermined period of time through the electrode unit; and a process of restoring to a state before the electrode unit encloses the tube in the body by controlling the manipulation unit.

The process of enclosing a tube in the body may include a process of identifying the tube in the body and a connective tissue and a sympathetic nerve tissue enclosing the tube in the body.

The process of enclosing a tube in the body may include a process of receiving information about a diameter of the tube in the body and bringing the electrode unit into close contact with an outer wall of the tube in the body based on the information about the diameter of the tube in the body.

The process of blocking a sympathetic nerve tissue may include a process of monitoring a temperature of the tube in the body.

In the process of blocking a sympathetic nerve tissue, a current may be transferred in order for the temperature of the tube in the body to be maintained between predetermined temperature values.

The method may further include a process of blocking the energy by manipulating the manipulation unit when an error occurs in the electrode apparatus.

The process of blocking a sympathetic nerve tissue may include a process of monitoring a contact state between the electrode unit and the tube in the body.

The method may further include a process of unwinding the electrode unit enclosing the tube in the body by manipulating the manipulation unit when the contact state between the electrode unit and the tube in the body is poor.

### Effects of the Invention

In a method of treating cardiac arrhythmia using an electrode apparatus according to an embodiment of the present disclosure, the electrode apparatus is inserted into a body and energy is transferred to a tube in the body through an electrode unit of the electrode apparatus to block a sympathetic nerve tissue. Thus, it is possible to inhibit the activity of the sympathetic nerve tissue distributed in the heart and thus treat atrial fibrillation.

The effects of the present disclosure are not limited to the aforementioned effects, and other effects, which are not mentioned above, will be apparent to a person with ordinary skill in the art from the following description.

### BRIEF DESCRIPTION OF THE DRAWINGS

FIG. 1 is a perspective view of an electrode apparatus for a method of treating cardiac arrhythmia using the electrode apparatus according to an embodiment of the present disclosure.

FIG. 2 illustrates an electrode unit of the electrode apparatus for the method of treating cardiac arrhythmia using the electrode apparatus according to an embodiment of the present disclosure.

FIG. 3 illustrates an energy generator for the method of treating cardiac arrhythmia using the electrode apparatus according to an embodiment of the present disclosure.

FIG. 4 is a flowchart showing the method of treating cardiac arrhythmia using the electrode apparatus according to an embodiment of the present disclosure.

FIG. 5 illustrates a state where the electrode unit of the electrode apparatus encloses a tube in the method of treating cardiac arrhythmia using the electrode apparatus according to an embodiment of the present disclosure.

FIG. 6 illustrates a state where energy is transferred to the tube through the electrode unit in the method of treating cardiac arrhythmia using the electrode apparatus according to an embodiment of the present disclosure.

FIG. 7A illustrates a process of preparing animals for animal test by using the method of treating cardiac arrhythmia using the electrode apparatus according to an embodiment of the present disclosure.

FIG. 7B is a flowchart showing a test procedure with animals by the method of treating cardiac arrhythmia using the electrode apparatus according to an embodiment of the present disclosure.

FIG. 7C shows examples of vagus nerve stimulation measurements.

FIG. 8A illustrates an atrial effective refractory period of a Sham group and an RDN group before vagus nerve stimulation.

FIG. 8B illustrates an atrial effective refractory period of the Sham group and the RDN group after vagus nerve stimulation.

FIG. 9A illustrates atrial fibrillation of the Sham group and the RDN group before vagus nerve stimulation.

FIG. 9B illustrates atrial fibrillation of the Sham group and the RDN group after vagus nerve stimulation.

FIG. 10A shows the kidney and renal artery isolated from a dead pig after completion of a test.

FIG. 10B is an enlarged view of the renal artery isolated from the dead pig after completion of the test.

FIG. 10C is a cross-sectional view of the renal artery isolated from a dead pig in the Sham group after completion of the test.

FIG. 10D is a cross-sectional view of the renal artery isolated from a dead pig in the RDN group after completion of the test.

FIG. 10E shows a tissue of the renal artery isolated from the dead pig in the Sham group after completion of the test.

FIG. 10F shows a tissue of the renal artery isolated from the dead pig in the RDN group after completion of the test.

### BEST MODE FOR CARRYING OUT THE INVENTION

Hereinafter, embodiments of the present disclosure will be described in detail with reference to the accompanying drawings so that a person having ordinary skill in the art can easily implement the present disclosure. However, the present disclosure may be implemented in various different forms and is not limited to the embodiments described herein. In the drawings, parts not related to the description are omitted for clarity, and like reference numerals designate like parts throughout the specification.

Throughout the specification, when a part is described as “including” a certain component, this means that other components may be further included rather than excluding other components, unless specifically stated otherwise. In addition, when a part is described as being “connected” to another part, this includes not only a case where the parts are directly connected, but also a case where the parts are connected through another member interposed therebetween and a case where the parts are electrically connected through another element interposed therebetween. Furthermore, when a member is described as being positioned “on” another member, this includes not only a case where the member is in contact with the other member, but also a case where another member is present between the two members. In addition, expressions such as “first,” “second,” and the like used herein may modify various components regardless of order and/or importance, and are used only to distinguish one component from another component, and do not limit the corresponding components, and do not necessarily imply different components. For example, a “first direction” and a “second direction” may indicate the same direction or different directions.

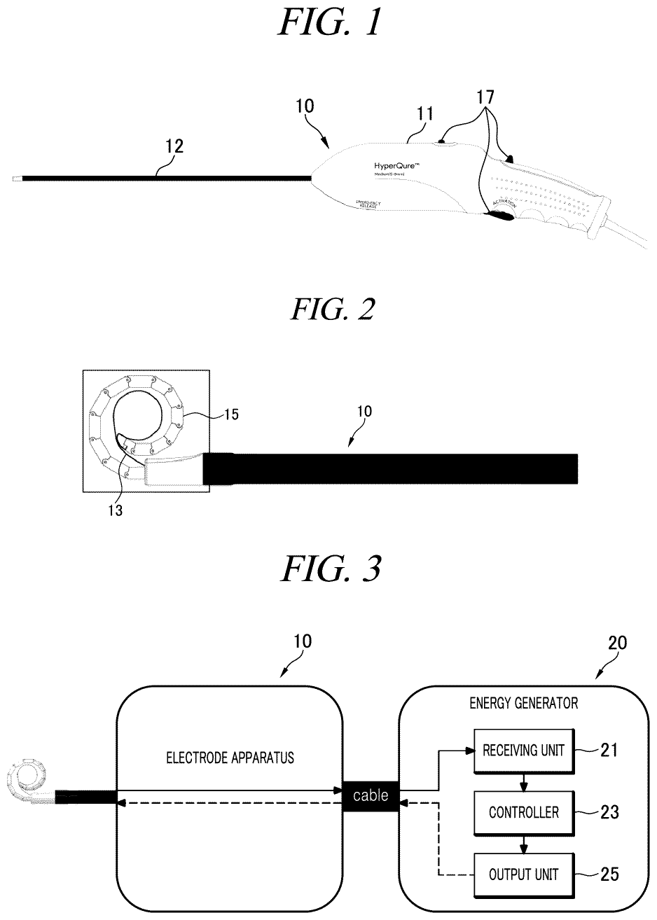

FIG. 1 is a perspective view of an electrode apparatus 10 for a method of treating cardiac arrhythmia using the electrode apparatus 10 according to an embodiment of the present disclosure, and FIG. 2 illustrates an electrode unit 13 of the electrode apparatus 10 for the method of treating cardiac arrhythmia using the electrode apparatus 10 according to an embodiment of the present disclosure.

Referring to FIG. 1 and FIG. 2, the electrode apparatus 10 for the method of treating cardiac arrhythmia using the electrode apparatus 10 according to an embodiment of the present disclosure includes a main body 11, the electrode unit 13, an electrode guide 15, and a manipulation unit 17.

The main body 11 may include a shaft 12 extending in one direction and a grip portion connected to the shaft 12 so as to be gripped by an operator. The components for driving and controlling the electrode unit 13 and the electrode guide may be located inside the main body 11.

The electrode unit 13 is formed to be drawn out from one end of the shaft 12 and configured to denervate or modulate at least a part of nerves distributed on a tissue including a tube V in the body depending on manipulation by the operator. The electrode unit 13 is accommodated inside the shaft 12 and when the electrode apparatus operates, the electrode unit 13 can be drawn out by means of the electrode guide which will be described later.

The electrode unit 13 may be made of a material, such as stainless steel or gold, which is harmless to the human body and conducts electricity well in order to block or denervate or control or modulate the nerves. Also, the electrode unit 13 may transfer various types of energy from an energy source generator. For example, the energy may include radio-frequency (RF) energy, electrical energy, laser energy, ultrasonic energy, high-intensity focused ultrasound energy, cryogenic energy, and other heat energy.

Further, the electrode unit 13 may be implemented as a flexible printed circuit board (PCB) for transferring RF energy, a transducer for transferring ultrasonic energy or a metal electrode for transferring high-voltage energy and thus may transfer energy to damage the nerves.

Also, the electrode unit 13 may include a sensor unit. For example, the sensor unit may be a thermocouple that measures a temperature by contact with the tube V in the body or the like. When neurotomy is performed with the electrode apparatus according to the present disclosure, the sensor unit can monitor a temperature of a treatment site.

Meanwhile, the electrode guide 15 functions to bring the electrode unit 13 into contact with the tube V in the body. The electrode guide 15 supports the electrode unit 13 and guides the electrode unit 13 to be in contact with the tube V in the body.

The electrode guide 15 of the present disclosure includes a plurality of joint units. The plurality of joint units forms a curved winding path to enclose the circumference of the tube V in the body with the electrode unit 13 interposed therebetween.

The manipulation unit 17 may include an electrode manipulation unit formed on the main body 11 so as to manipulate energy transfer to the electrode unit 13, and a guide manipulation unit formed on the main body 11 so as to manipulate an operation of the electrode guide.

FIG. 3 illustrates an energy generator 20 for the method of treating cardiac arrhythmia using the electrode apparatus 10 according to an embodiment of the present disclosure.

In the electrode apparatus 10, a signal value of a temperature of the tube V in the body measured by the electrode unit 13 is pre-processed by a signal processor, and pre-processed temperature data are transmitted to the energy generator 20 through a cable.

Herein, referring to FIG. 3, the energy generator 20 may include a receiving unit 21, a controller 23, and an output unit 25. However, these components are illustrated as example components which can be controlled by the energy generator 20.

The energy generator 20 outputs energy to an electrode apparatus for nerve denervation or modulation in vivo. For example, the energy generator 20 receives monitored temperature data of the tube V in the body, which is a procedure target, from the sensor unit of the electrode apparatus 10 through the cable. The energy generator 20 outputs energy to the electrode apparatus 10 based on the received temperature data.

Herein, the energy generator 20 can control an output intensity of energy based on a temperature variance of the tube V in the body. Hereafter, the components of the energy generator 20 will be described in detail.

The receiving unit 21 receives the temperature data of the tube V in the body from the electrode apparatus 10. For example, the receiving unit 21 receives a temperature signal of the tube V in the body measured by the sensor unit of the electrode apparatus through the cable. For example, the receiving unit 21 pre-processes the received temperature signal of the tube V in the body through conversion of the temperature signal into a temperature value and transmits the temperature value to the controller 23.

The controller 23 calculates a temperature value of the tube V in the body based on the received temperature data. For example, the controller 23 may calculate a temperature variance of the tube V in the body by comparing temperature data of the tube V in the body received from the electrode apparatus at a regular interval. Further, the controller 23 may calculate a temperature variance of the tube by comparing temperature data of the tube obtained before and after output of energy to the electrode apparatus 10.

The controller 23 determines an intensity of energy through PID control based on the temperature variance of the tube. That is, the controller 23 determines PID constants for PID control based on the temperature variance of the tube. Herein, the PID constants include a proportional constant, an integral constant, and a derivative constant.

Specifically, the controller 23 compares the temperature variance of the tube with a predetermined threshold value. When the temperature variance of the tube is lower than the predetermined threshold value based on the result of the comparison, the controller 23 sets the PID constants as predetermined constants.

The predetermined threshold value is set to include a plurality of values at a regular interval, and the predetermined PID constants are differently set for each interval. For example, the threshold value is set to include a plurality of values at a regular interval of 0.5° C. For example, the threshold value may be set to 0.5° C., 1.0° C., 1.5° C., or 2.0° C. Herein, when the threshold value is 0.5° C., the PID constants are set to KP1, KI1, and KD1; when the threshold value is 1.0° C., the PID constants are set to KP2, KI2, and KD2; when the threshold value is 1.5° C., the PID constants are set to KP3, KI3, and KD3; and when the threshold value is 2.0° C., the PID constants are set to KP4, KI4, and KD4.

Further, the threshold value may be determined based on an object including the tube, a type of the tube, and a location of the tube. That is, the threshold value may be set according to the characteristics of the procedure target.

The output unit 25 outputs the energy to the electrode apparatus based on the determined intensity of energy. For example, the output unit 25 outputs the energy to the electrode apparatus through the cable at the intensity set according to the PID control constants by the controller 23.

As described above, the energy generator 20 can regulate the PID control constants depending on the temperature variance of the tube V in the body during a surgical procedure. Therefore, the energy generator 20 can perform a surgical procedure reflecting the characteristics of a body that vary among individuals. Accordingly, it is possible to perform denervation more safely and precisely.

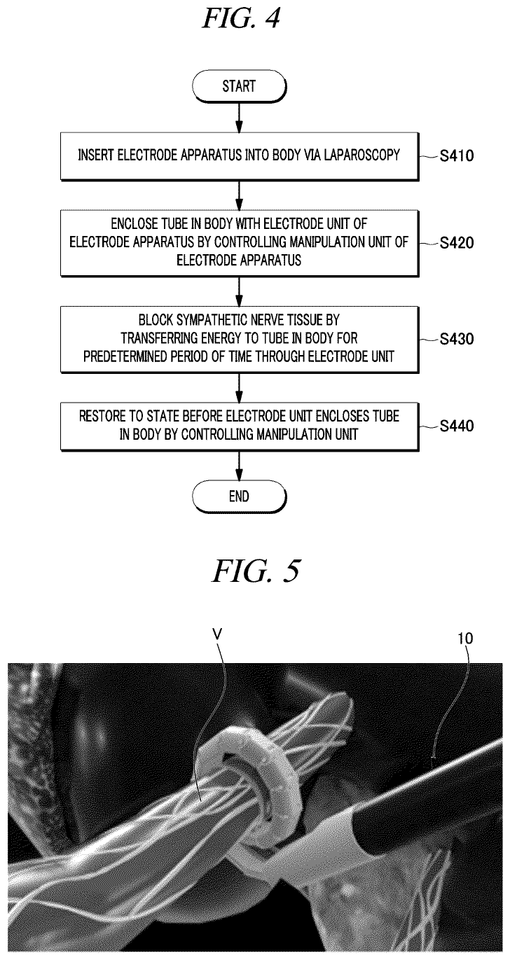

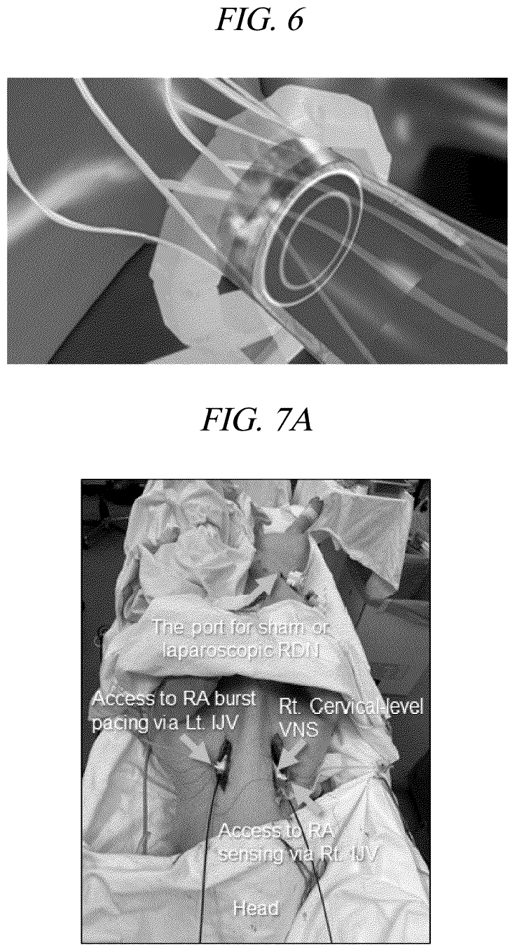

FIG. 4 is a flowchart showing the method of treating cardiac arrhythmia using the electrode apparatus 10 according to an embodiment of the present disclosure, FIG. 5 illustrates a state where the electrode unit 13 of the electrode apparatus 10 encloses a tube in the method of treating cardiac arrhythmia using the electrode apparatus 10 according to an embodiment of the present disclosure, and FIG. 6 illustrates a state where energy is transferred to the tube through the electrode unit 13 in the method of treating cardiac arrhythmia using the electrode apparatus 10 according to an embodiment of the present disclosure.

Referring to FIG. 4, the method of treating cardiac arrhythmia using the electrode apparatus 10 according to an embodiment of the present disclosure includes the processes time-sequentially performed by using the manipulation unit 17 and the electrode unit 13 included in the electrode apparatus 10.

In a process S110, laparoscopy may be performed to insert the electrode apparatus 10 into a body.

Before the electrode apparatus 10 is inserted into the body, an adipose tissue around the tube V in the body is ablated with an ablation tool or an electrocautery to easily perform denervation.

Also, before the electrode apparatus 10 is inserted into the body, a diameter of the tube V in the body is measured via Computed Tomography (CT) and laparoscopy to set the electrode unit 13 suitable for the diameter of the tube V in the body.

For example, the range of a diameter that the electrode unit 13 can enclose may be one of from 2 mm to 5 mm, from 5 mm to 8 mm, and from 8 mm to 11 mm, and the electrode unit 13 may be used corresponding to the diameter of the tube V in the body.

Herein, the range of the diameter that the electrode unit 13 can enclose includes the diameter of the tube V in the body as well as the thickness of the tissue remaining around the tube V in the body. For example, if the diameter of the tube V in the body is 4 mm and the thickness of the tissue remaining around the tube V in the body is 2 mm, the range of the diameter that the electrode unit 13 can enclose may be set to from 5 mm to 8 mm and the electrode unit 13 corresponding thereto may be used.

In a process S120, the electrode unit 13 of the electrode apparatus 10 may enclose the tube V in the body by controlling the manipulation unit 17 of the electrode apparatus 10.

Herein, the process of enclosing the tube V in the body may include a process of identifying the tube V in the body and a connective tissue and a sympathetic nerve tissue enclosing the tube V in the body.

For example, the tube V in the body and the connective tissue and the sympathetic nerve tissue enclosing the tube V in the body are identified before the electrode unit 13 encloses the tube V in the body, and, thus, the electrode unit 13 may be controlled through the manipulation unit 17 to closely enclose the tube V in the body corresponding to the thickness of the tube V in the body and the tissue remaining around the tube V in the body.

Also, in the process of enclosing the tube V in the body, information about the diameter of the tube V in the body may be received and the electrode unit 13 may be brought into close contact with an outer wall of the tube V in the body based on the information about the diameter of the tube V in the body.

Herein, in the process of bringing the electrode unit 13 into close contact with an outer wall of the tube V in the body, denervation may be performed by bringing the electrode unit 13 into close contact with the tube V in the body by an external force or a tensile force and transferring energy to damage the nerves.

In a process S130, a sympathetic nerve tissue may be blocked by transferring energy to the tube V in the body for a predetermined period of time through the electrode unit 13.

For example, if the predetermined period of time is 70 seconds in the process of blocking the sympathetic nerve tissue, the sympathetic nerve tissue may be blocked by transferring energy for the predetermined period of time. However, the present disclosure is not limited thereto. The predetermined period of time may vary depending on the diameter of the tube V in the body, the location of the tube V in the body, the thickness of the connective tissue enclosing the tube V in the body, and the sympathetic nerve tissue.

After the predetermined period of time has passed, energy generated by the electrode unit 13 is automatically cut off and the electrode unit 13 enclosing the tube V in the body can be transitioned to the state before enclosing the tube V in the body through the manipulation unit 17.

The process of blocking the sympathetic nerve tissue may include a process of monitoring a temperature of the tube V in the body.

In the process of monitoring a temperature of the tube V in the body, the temperature of the tube V in the body may be monitored by the sensor unit formed in the electrode unit 13, and the sensor unit can suppress damage to the tube V in the body in advance by monitoring an abnormal increase in the temperature of the tube V in the body.

In the process of blocking the sympathetic nerve tissue, output of energy may be regulated through the energy generator 20 in order for the temperature of the tube V in the body to be maintained at a predetermined temperature value.

For example, when the predetermined temperature value is 50° C., an output intensity of energy may be controlled through the energy generator 20 in order for the temperature of the tube V in the body to be maintained at the predetermined temperature value (50° C.) and energy may be transferred to the tube V in the body through the electrode unit 13. Herein, the temperature of the tube V in the body may be monitored in real time by the sensor unit formed in the electrode unit 13, and when the temperature of the tube V in the body is higher than the predetermined temperature value (50° C.), the predetermined temperature value (50° C.) can be maintained with a minimum output of energy. However, the present disclosure is not limited thereto. The predetermined temperature value may vary depending on the diameter of the tube V in the body, the location of the tube V in the body, the thickness of the connective tissue enclosing the tube V in the body, and the sympathetic nerve tissue.

Meanwhile, the method of treating cardiac arrhythmia may further include a process of blocking the energy by manipulating the manipulation unit 17 when an error occurs in the electrode apparatus 10.

For example, when an error occurs in the electrode unit 13, such as energy transfer to the tube V in the body for longer than the predetermined period of time or to higher than the predetermined temperature value, the energy may be blocked by manipulating the manipulation unit 17.

Meanwhile, the process of blocking the sympathetic nerve tissue may include a process of monitoring a contact state between the electrode unit 13 and the tube V in the body.

Due to the process of monitoring a contact state between the electrode unit 13 and the tube V in the body, it is possible to check the contact state between the electrode unit 13 and the tube V in the body that the operator has difficulty in checking with the naked eye and thus possible to accurately and precisely perform a surgical procedure.

The method of treating cardiac arrhythmia may further include a process of unwinding the electrode unit 13 enclosing the tube V in the body by manipulating the manipulation unit 17 when the contact state between the electrode unit 13 and the tube V in the body is poor.

For example, due to the process of monitoring a contact state between the electrode unit 13 and the tube V in the body, it is possible to determine whether the contact state between the electrode unit 13 and the tube V in the body is poor and also possible to unwind the electrode unit 13 enclosing the tube V in the body by manipulating the manipulation unit 17.

After the sympathetic nerve tissue is blocked by transferring the energy through the electrode unit 13, the manipulation unit 17 may be controlled to transition the electrode unit 13 to the state before enclosing the tube V in the body.

Meanwhile, the present inventors conducted an animal test of the method of treating cardiac arrhythmia using the electrode apparatus 10 according to an embodiment of the present disclosure. As a result, the present inventors verified a decrease in induction of atrial fibrillation (AF) and an increase in atrial effective refractory period (AERP).

The present test was conducted to verify acute effects of the method of treating cardiac arrhythmia using the electrode apparatus 10 according to an embodiment of the present disclosure on regulation of induction of AF in pig models.

In this test, a total of 14 pig models were used, with 2 pigs used to test and establish protocols for vagus nerve stimulation and electrophysiological studies. Also, 3 pigs were used to perform the method of treating cardiac arrhythmia using the electrode apparatus 10 according to an embodiment of the present disclosure, and 9 pigs were randomly assigned to either a Sham group or an RDN (Renal Denervation) group.

Before the test, the pigs were fasted for 12 hours, and general anesthesia was induced by intramuscular injection of Zoletil (5 mg/kg) and Xylazine (2 mg/kg). After general anesthesia was induced, endotracheal intubation was performed using an endotracheal tube and anesthesia was maintained with 2.0-2.5% isoflurane during the test. Mechanical ventilation was adjusted to maintain oxygen saturation at 98% or more and CO₂ at 38-40 mmHg. Electrodes were attached to the limbs to monitor electrocardiograms during the test, and the chest and abdomen were disinfected with povidone-iodine and covered with sterile drapes.

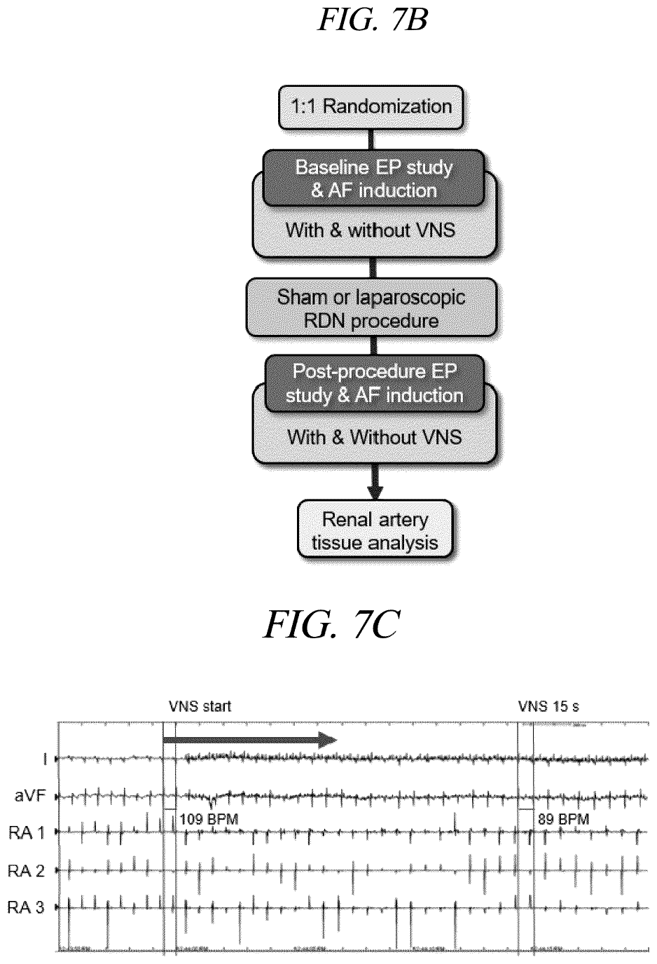

FIG. 7A illustrates a process of preparing animals for animal tests by using the method of treating cardiac arrhythmia using the electrode apparatus 10 according to an embodiment of the present disclosure, FIG. 7B is a flowchart showing a test procedure with animals by the method of treating cardiac arrhythmia using the electrode apparatus 10 according to an embodiment of the present disclosure, and FIG. 7C shows examples of vagus nerve stimulation measurements.

Referring to FIG. 7B, the test may include an animal preparation stage, an AERP measurement stage, an AF induction measurement stage, a vagus nerve stimulation (VNS) stage, a laparoscopic RDN stage, and a tissue analysis stage.

Referring to FIG. 7A, the animal preparation stage involved a vertical skin incision between the sternohyoid muscle and sternocleidomastoid muscle located on both sides of the trachea, and dissection of subcutaneous tissues to expose the internal jugular vein and carotid artery. The right and left jugular veins were punctured with 7-Fr sheaths, and a catheter was inserted into the right atrium through the internal jugular vein. The right jugular catheter was used to record intracardiac electrocardiograms, while the left jugular catheter was used to stimulate the right atrium.

A pacing output at an interval of 400 ms was changed from 10 V to 2 V, and the left catheter was adjusted in location to maintain stable stimulation. The stability of the stimulation was verified by using both surface and intracardiac electrocardiograms, and the right carotid artery was exposed and contracted to locate the right vagus nerve.

During the AERP measurement stage, a contraction threshold value of the right atrium was tested by reducing a pacing output from 10 V to 1 V. The AERP was measured by using an S1-S2 protocol. Herein, S1 is a reference stimulus consisting of 8 consecutive stimuli, and S2 is a stimulus given after S1 to measure the AERP. To measure the AERP, an S2 stimulus interval was reduced by 10 ms decrements, starting from 400 ms. A minimum stimulus interval from when the S2 stimulus interval reached the AERP until no effective stimulus was elicited in the atrium was set as the AERP. The protocol was repeated three times to confirm the AERP, and atrial pacing and intracardiac electrocardiograms were recorded on a Prucka Cardiolab EP System (GE Medical Systems, Fairfield, CT, USA).

During the AF induction measurement stage, burst stimulation was applied to the right atrium at an interval of 100 ms and a pulse width of 1.0 ms for 60 seconds. AF induction was repeated more than 10 times with a 60-second rest interval, and the induced AF episodes were confirmed from surface and intracardiac electrocardiogram recordings. The duration of each AF episode was measured. The AERP measurement and AF induction measurement were conducted on each pig included in the Sham group or the RDN group before and after the test, and repeated regardless of vagus nerve stimulation.

During the VNS stage, an electrocardiogram electrode was placed and fixed on the right cervical vagus nerve located below the carotid artery. VNS was performed with a stimulator at a pulse width of 0.2 ms and a frequency of 20 Hz. A VNS output was adjusted between 2 V an 10 V to reduce the heart rate by 10% without causing mechanical instability. Accordingly, as shown in FIG. 7C, VNS measurements were recorded.

During the laparoscopic RDN stage, after draping, a needle was inserted laterally 5 cm from the uterus or 2 cm below the umbilicus and CO₂ was injected into the abdominal cavity. The renal artery was exposed by dissecting the surrounding soft tissue, and renal denervation (RDN) was performed around the renal artery by using the electrode apparatus 10 of the present disclosure. After the renal artery was enclosed with the electrode unit 13 of the electrode apparatus 10, bipolar radiofrequency energy was delivered for 70 seconds at a constant temperature of 65° C. After RDN, the surrounding tissue was sutured and CO₂ was removed from the abdominal cavity. All the processes of the Sham procedure except for the delivery of radiofrequency energy were performed in the same manner as in the RDN group.

During the tissue analysis stage, potassium chloride was administered intravenously to the pigs after the procedure, and the renal arteries were harvested. The renal arteries were fixed in 10% formalin and stained with hematoxylin and eosin. Also, 4 μm thick horizontal sections were prepared from the proximal and distal cut ends of the renal arteries by using paraffin, and immunohistochemical staining was performed by using a monoclonal mouse antibody to visualize the renal sympathetic nerves. The nerve fibers along the outer membrane of the renal artery were examined for comparison between the Sham group and the RDN group.

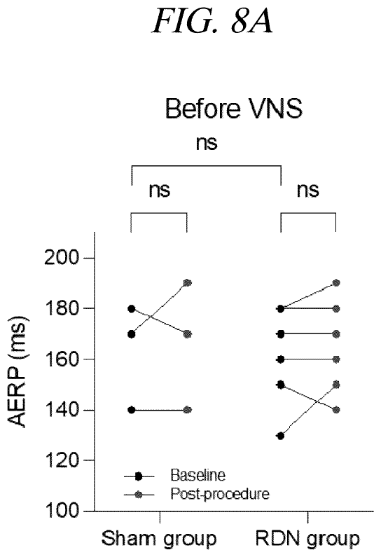

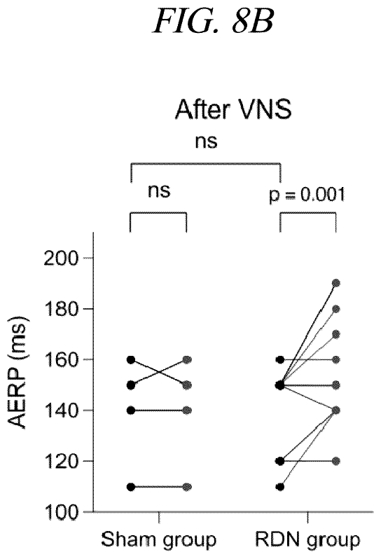

FIG. 8A illustrates an atrial effective refractory period of a Sham group and an RDN group before vagus nerve stimulation, and FIG. 8B illustrates an atrial effective refractory period of the Sham group and the RDN group after vagus nerve stimulation.

Referring to FIG. 8A and FIG. 8B as a result of the test, the baseline AERP before the VNS stage was 170 (140 to 180) ms in the Sham group and 170 (160 to 180) ms in the RDN group. After the VNS stage, a decrease in the AERP was observed regardless of RDN or group, and the AERP was decreased by 30 ms in each of the two groups. A similar trend was also observed in the AERP after the RDN procedure, with a decrease by 30 ms and 10 ms. Before the VNS stage, there was no difference between the baseline AERP and the AERP measured after the RDN procedure in both the Sham group and the RDN group. However, after the VNS stage, the AERP measured after the RDN procedure was increased by 20 ms. In contrast, no difference was observed in the AERP after the Sham procedure.

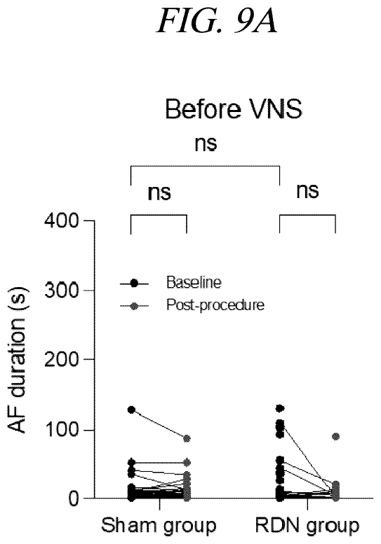

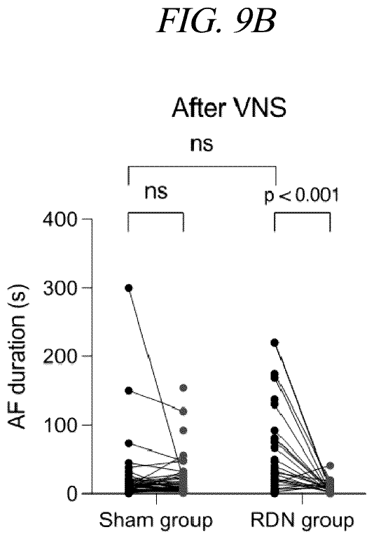

FIG. 9A illustrates atrial fibrillation of the Sham group and the RDN group before vagus nerve stimulation, and FIG. 9B illustrates atrial fibrillation of the Sham group and the RDN group after vagus nerve stimulation.

Further, referring to FIG. 9A and FIG. 9B as a result of the test, the Sham procedure did not significantly change the duration of AF in the Sham group regardless of VNS, whereas in the RDN group, the duration of AF was decreased after the RDN procedure prior to VNS. Furthermore, in the RDN group, the duration of AF after the VNS stage and the RDN procedure was greatly decreased.

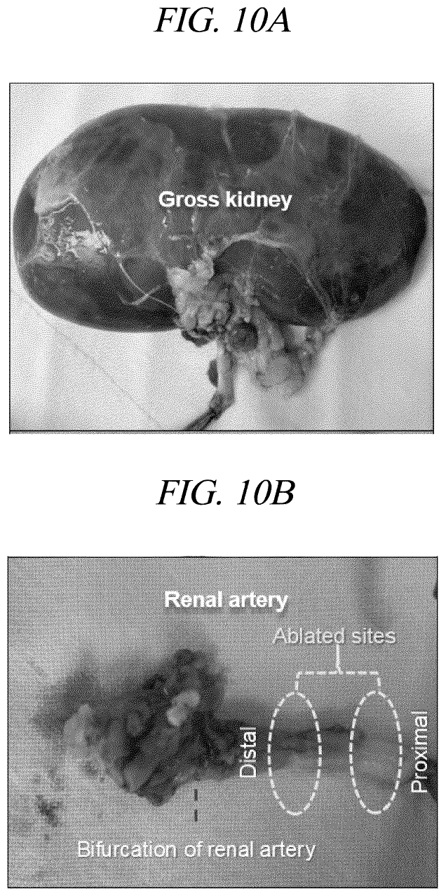

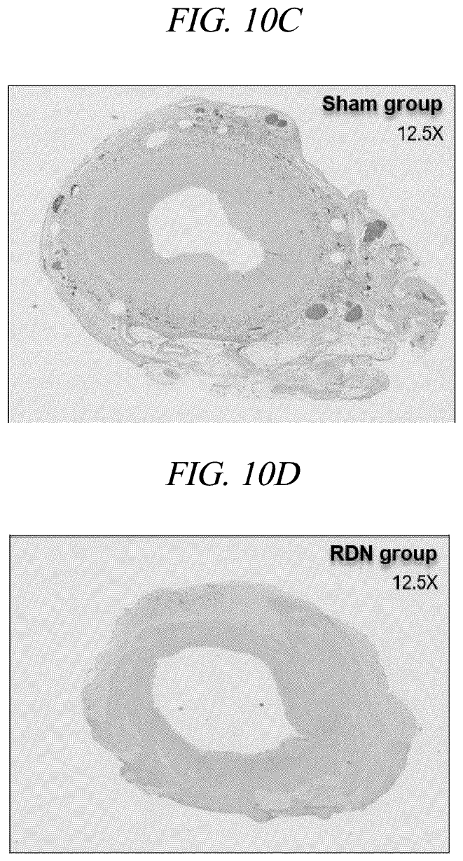

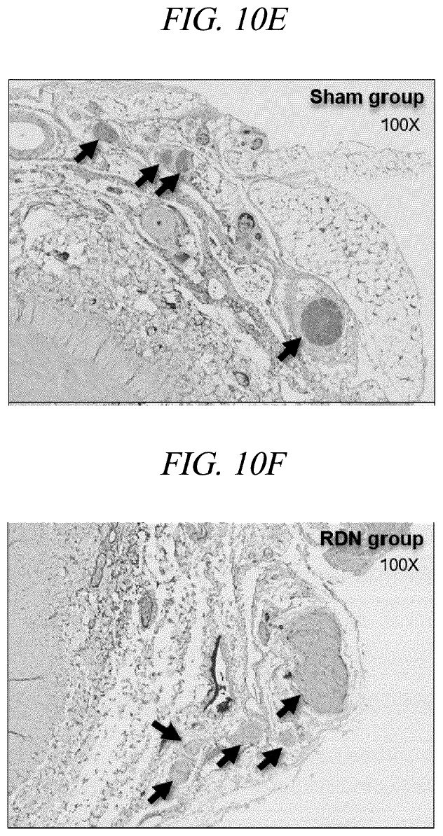

FIG. 10A shows the kidney and renal artery isolated from a dead pig after completion of a test, FIG. 10B is an enlarged view of the renal artery isolated from the dead pig after completion of the test, FIG. 10C is a cross-sectional view of the renal artery isolated from a dead pig in the Sham group after completion of the test, FIG. 10D is a cross-sectional view of the renal artery isolated from a dead pig in the RDN group after completion of the test FIG. 10E shows a tissue of the renal artery isolated from the dead pig in the Sham group after completion of the test, and FIG. 10F shows a tissue of the renal artery isolated from the dead pig in the RDN group after completion of the test.

Further, referring to FIG. 10A and FIG. 10B as a result of the test, it was confirmed that the subcutaneous tissue around the renal artery had been dissected along the ablated area. Referring to FIG. 10C and FIG. 10D, the cross-sectional slides of the renal artery confirmed that there was no significant vascular malformation or damage to the tunica media caused by a laparoscopic RDN procedure. Furthermore, referring to FIG. 10E and FIG. 10F, the renal sympathetic nerves were found to be destroyed in the RDN group.

These results suggest that the method of treating cardiac arrhythmia using the electrode apparatus 10 according to an embodiment of the present disclosure can reduce the occurrence of AF, particularly in vagally mediated types. Moreover, the method of treating cardiac arrhythmia using the electrode apparatus 10 according to an embodiment of the present disclosure can effectively block the renal sympathetic nerves while preserving the renal artery structure without causing severe vascular damage.

As described above, in the method of treating cardiac arrhythmia using the electrode apparatus 10 according to an embodiment of the present disclosure, the electrode apparatus 10 is inserted into the body and energy is transferred to the tube V in the body through the electrode unit 13 of the electrode apparatus 10 to block a sympathetic nerve tissue. Thus, it is possible to inhibit the activity of the sympathetic nerve tissue distributed in the heart and thus treat AF.

The tube V in the body and the connective tissue and the sympathetic nerve tissue enclosing the tube V in the body are identified before the electrode unit 13 encloses the tube V in the body, and, thus, the electrode unit 13 may be controlled through the manipulation unit 17 to closely enclose the tube V in the body corresponding to the thickness of the tube V in the body and the tissue remaining around the tube V in the body.

Also, due to the process of monitoring a temperature of the tube V in the body, the temperature of the tube V in the body can be monitored by the sensor unit formed in the electrode unit 13, and the sensor unit can suppress damage to the tube V in the body in advance by monitoring an abnormal increase in the temperature of the tube V in the body. Also, the energy generator 20 regulates output of energy to maintain the temperature of the tube V in the body at a predetermined temperature value, and, thus, it is possible to suppress damage to the tube V in the body caused by high temperature.

The above description of the present disclosure has been provided for illustrative purposes. A person having ordinary skill in the art will understand that various modifications may be made in other specific forms without departing from the technical spirit or essential features of the present disclosure. Therefore, the embodiments described above should be understood in all respects as illustrative and not limiting. For example, each component described as being implemented in a single form may be implemented in a distributed manner, and likewise, components described as being distributed may be implemented in a combined form.

Further, the scope of the present disclosure should be defined by the claims set forth below rather than by the foregoing detailed description, and all changes or modifications derived from the meaning and scope of the claims and their equivalents should be construed as being included within the scope of the present disclosure.

We claim:

1. A method of treating cardiac arrhythmia using an electrode apparatus, comprising:
a process of inserting the electrode apparatus into a body via laparoscopy;
a process of enclosing a tube in the body with an electrode unit of the electrode apparatus by controlling a manipulation unit of the electrode apparatus;
a process of blocking a sympathetic nerve tissue by transferring energy to the tube in the body for a predetermined period of time through the electrode unit; and
a process of restoring to a state before the electrode unit encloses the tube in the body by controlling the manipulation unit.

2. The method of treating cardiac arrhythmia using an electrode apparatus of claim 1,
wherein the process of enclosing a tube in the body includes a process of identifying the tube in the body, a connective tissue and a sympathetic nerve tissue that enclose the tube in the body.

3. The method of treating cardiac arrhythmia using an electrode apparatus of claim 1
wherein the process of enclosing a tube in the body includes a process of receiving information about a diameter of the tube in the body and bringing the electrode unit into close contact with an outer wall of the tube in the body based on the information about the diameter of the tube in the body.

4. The method of treating cardiac arrhythmia using an electrode apparatus of claim 1,
wherein the process of blocking a sympathetic nerve tissue includes a process of monitoring a temperature of the tube in the body.

5. The method of treating cardiac arrhythmia using an electrode apparatus of claim 4,
wherein in the process of blocking a sympathetic nerve tissue, energy is transferred in order for the temperature of the tube in the body to be maintained at a predetermined temperature value.

6. The method of treating cardiac arrhythmia using an electrode apparatus of claim 1, further comprising:
a process of blocking transfer of the energy by manipulating the manipulation unit when an error occurs in the electrode apparatus.

7. The method of treating cardiac arrhythmia using an electrode apparatus of claim 1,
wherein the process of blocking a sympathetic nerve tissue includes a process of monitoring a contact state between the electrode unit and the tube in the body.

8. The method of treating cardiac arrhythmia using an electrode apparatus of claim 7, further comprising:
a process of unwinding the electrode unit enclosing the tube in the body by manipulating the manipulation unit when the contact state between the electrode unit and the tube in the body is poor.
